SEQUENCE LISTING (5 sequences)

SEQ ID NO: 1 - DNA, 22 bp, Artificial
  Primer
cgcgaaggggccaccaaagaac

SEQ ID NO: 2 - DNA, 24 bp, Artificial
  Primer
tccccagaaagcaagcaaaagact

SEQ ID NO: 3 - DNA, 27 bp, Artificial
  Primer
cctctccaatcttagtcggtcctttct

SEQ ID NO: 4 - DNA, 24 bp, Artificial
  Primer
cctaccggtggatgtggaatgtgt

SEQ ID NO: 5 - DNA, 24 bp, Artificial
  Primer
agccccctagcgtttcctctgtga

(19) United States
(12) Patent Application Publication
(10) Pub. No.: US 2006/0241073 A1
(43) Pub. Date: Oct. 26, 2006

(54) Means and methods for counteracting fatty acid accumulation

(76) Inventor: Ronaldus Joannes Adrianus Wanders, Hilversum (NL)

Correspondence Address: TRASK BRITT, P.O. BOX 2550, SALT LAKE CITY, UT, 84110, US

(21) Appl. No.: 11/110,989

(22) Filed: Apr. 20, 2005

(51) Int. Cl. A61K 48/00; A61K 38/44

(52) U.S. Cl. 514/44; 424/94.4

(57) ABSTRACT

The invention provides a method for, at least, in part, counteracting a disease involving accumulation of a fatty acid, the method comprising administering a compound which is capable of inducing and/or upregulating omega-oxidation of the fatty acid, or whose metabolite is capable of inducing and/or upregulating omega-oxidation of the fatty acid, to a subject suffering from, or at risk of suffering from, the disease.

### TECHNICAL FIELD

The invention relates to the field of medicine. More specifically, the invention relates to the treatment and diagnosis of diseases involving accumulation of fatty acids

### BACKGROUND

Fatty acids are an important source of energy. In an organism, fatty acids are constantly broken down and converted into other metabolites. These degradation processes involve the action of a wide variety of enzymes. Most linear fatty acids are degraded by a beta-oxidation pathway in peroxisomes or mitochondria. During beta-oxidation, a fatty acid is shortened by two carbon atoms at the carboxyl end to produce its n-2 analogue.

In general, branched chain fatty acids are also degraded by a beta-oxidation pathway in peroxisomes and mitochondria. However, this is only true for 2-methyl branched chain fatty acids. 3-Methyl branched fatty acids cannot be degraded by a beta-oxidation pathway because the methyl group at the third carbon atom blocks beta-oxidation. Degradation of 3-methyl branched fatty acids, therefore, proceeds primarily via alpha-oxidation. During alpha-oxidation, a fatty acid is shortened by one carbon atom at the carboxyl end to produce its n-1 analogue. The resulting fatty acid has a 2-methyl group and can be further degraded by beta-oxidation.

A defect in a fatty acid degradation pathway often results in accumulation of a fatty acid or a metabolite, thereof. A defective pathway is, for instance, caused by the absence of an (active) enzyme involved in the degradation of a fatty acid or a metabolite, thereof. Other causes of defective fatty acid degradation pathways, for instance, comprise the absence of an (active) protein involved in the transportation of a fatty acid or a metabolite, thereof, to a compartment such as a mitochondrion or a peroxisome where degradation should take place. In such a case, a fatty acid or metabolite is not capable of reaching the enzyme(s) that should degrade it. As a result, accumulation of at least one fatty acid or metabolite takes place.

Accumulation of fatty acids or metabolites, thereof, over time is mostly harmful for an organism. A wide variety of disorders is known involving the accumulation of one or several fatty acids or metabolites, thereof. Examples of such disorders are diabetes mellitus, peroxisomal fatty acid oxidation deficiencies, and mitochondrial fatty acid oxidation deficiencies.

Progression of disorders involving the accumulation of fatty acids is often prevented by avoiding the intake of products that comprise, or are converted into, fatty acids that cannot be degraded. Currently, it is not possible to counteract fatty acid accumulation disorders by administration of a missing protein to a patient suffering from such disorder. Although other accumulation diseases like Gaucher and Fabry disease are treated by administration of a missing enzyme (also called enzyme replacement therapy), this is not yet possible for fatty acid accumulation diseases.

Reasons for this are that the required protein, such as an enzyme capable of catalyzing alpha or beta oxidation of a fatty acid, is not always available, or that a protein does not arrive at the right location within an organism. For instance, administered enzymes are often transported into lysosomes, where they are destroyed.

It is an object of the present invention to provide an alternative method for, at least, in part, counteracting a disorder involving the accumulation of at least one fatty acid.

The invention provides a method for, at least, in part, counteracting a disease involving accumulation of at least one fatty acid, the method comprising administering a compound capable of inducing and/or upregulating omega-oxidation of the fatty acid to a subject suffering from, or at risk of suffering from, the disease. In one embodiment, a compound whose metabolite is capable of inducing and/or upregulating omega-oxidation of the fatty acid is administered to the subject.

According to the present invention, instead of administering an enzyme capable of catalyzing alpha-oxidation or beta-oxidation of a fatty acid, it is possible to use an alternative pathway in order to degrade fatty acids. In a method of the invention, degradation of a fatty acid via omega-oxidation is induced and/or enhanced. The resulting metabolite is, subsequently, further degraded. As a result, accumulation of a fatty acid is, at least, in part, diminished. A method of the invention is, thus, suitable for preventing progression of a disease involving fatty acid accumulation. Moreover, it has become possible to degrade fatty acids which are already accumulated within an organism. Hence, a method of the invention is also suitable for, at least, in part, diminishing the amount of fatty acid that has built up.

In an omega-oxidation pathway, the carbon atom at the omega-end of a fatty acid is hydroxylated by a member of the cytochrome P450 enzyme family. This hydroxylated fatty acid is then converted into an aldehyde by an alcohol dehydrogenase, and, subsequently, this aldehyde is converted into a carboxyl group by an aldehyde dehydrogenase. As a consequence, the final product of the pathway is a dicarboxylic fatty acid, which can be degraded further within an organism by other pathways, for instance, by beta-oxidation from the omega-end.

Cytochrome P450 enzymes naturally occur within organisms. The cytochrome P450 superfamily is highly diverse. Hundreds of P450 sequences are known. They are abundantly present within an organism: most cells comprise one or several cytP450 enzymes. Human individuals possess more than 50 different cytochrome P450 enzymes which are involved in a variety of functions, such as drug metabolism, blood hemostasis, cholesterol biosynthesis, and steroidogenesis. Although cytochrome P450 enzymes are capable of catalyzing omega-oxidation of fatty acids, they do not naturally catalyze this reaction to a sufficient extent to compensate for a failing fatty acid degradation pathway. According to the invention, it is, however, possible to induce and/or enhance an omega-oxidation capacity of at least one cytochrome P450 enzyme such that a disorder involving accumulation of at least one fatty acid is, at least, in part, counteracted. Most cytochrome P450 enzymes are present in the endoplasmic reticulum. They are capable of catalyzing fatty acids that are present in the cytosol. Hence, the amount and/or omega-oxidation capacity of at least one cytochrome P450 enzyme, which is present in the endoplasmic reticulum is, preferably, induced and/or enhanced in order to degrade a fatty acid which is present in the cytosol. Some cytochrome P450 enzymes are present within mitochondria. The amount and/or omega-oxidation capacity of at least one of these P450 enzymes is, therefore, preferably, induced and/or enhanced in order to degrade a fatty acid, which is present in mitochondria. As used herein, a cytochrome P450 enzyme is also referred to as a cytP450.

By, at least, in part, counteracting a disease involving accumulation of a fatty acid is herein meant, that a method is performed which results in a smaller amount of accumulated fatty acid in an organism as compared to the amount of accumulated fatty acid that would have been present in the organism if the method had not been performed.

A compound capable of inducing and/or upregulating omega-oxidation of a fatty acid is defined herein as:

a compound whose presence in an organism results in an enhanced level of omega-oxidation of a given fatty acid as compared to the level of omega-oxidation of the fatty acid that would have been present in the organism, if the compound had not been present, or

a compound which is converted in vivo into at least one metabolite whose presence in an organism results in an enhanced level of omega-oxidation of a given fatty acid as compared to the level of omega-oxidation of the fatty acid that would have been present in the organism, if the metabolite had not been present.

A metabolite of a compound is defined as a molecule which is formed when the compound is processed in vivo. After administration of a compound such as, for instance, a prodrug to an animal, the compound is sometimes altered within the animal. The compound is, for instance, cleaved. As another example, the compound, or a metabolite, thereof, is modified by conjugation with an endogenous molecule such as, for instance, glucuronic acid, glutathione and/or sulfate. A metabolite resulting from such modification may, subsequently, be cleaved, and/or a cleavage product may, subsequently, be modified. Any product resulting from in vivo processing of a compound is called herein, a metabolite of the compound. If the metabolite is capable of inducing and/or upregulating omega-oxidation of a fatty acid, it is suitable for a method of the present invention. In that case, it is possible to administer the compound and/or at least one suitable metabolite. A compound capable of inducing and/or upregulating omega-oxidation of a fatty acid, therefore, encompasses compounds that are converted in vivo into at least one metabolite, which is capable of inducing and/or upregulating omega-oxidation of a fatty acid.

Induction and/or upregulation of omega-oxidation of a fatty acid results in less accumulation of the fatty acid. A compound which is capable of inducing and/or upregulating omega-oxidation of a fatty acid, or whose metabolite is capable of inducing and/or upregulating omega-oxidation of a fatty acid, is, therefore, suitable for, at least, in part, counteracting a disease involving accumulation of the fatty acid. The invention, therefore, provides a use of a compound which is capable of upregulating omega-oxidation of a fatty acid, or whose metabolite is capable of inducing and/or upregulating omega-oxidation of a fatty acid, for the manufacture of a medicament for, at least, in part, treating a disease involving accumulation of the fatty acid.

In a preferred embodiment, a method or a use according to the invention is provided, wherein the disease comprises diabetes mellitus. In another preferred embodiment, the disease comprises a peroxisomal and/or mitochondrial fatty acid oxidation deficiency like, but not restricted to, camitine palmitoyl transferase 1 and 2 deficiency, very-long-chain acyl-CoA dehydrogenase deficiency, medium-chain acyl-CoA dehydrogenase deficiency, long-chain 3-hydroxy acyl-CoA dehydrogenase deficiency, mitochondrial trifunctional protein deficiency, Refsum disease, and/or X-linked adrenoleukodystrophy. In a most preferred embodiment, the disease comprises diabetes mellitus, a mitochondrial fatty acid oxidation deficiency, Refsum disease and/or X-linked adrenoleukodystrophy.

Omega-oxidation of a given fatty acid is induced and/or upregulated in various ways. In one embodiment, omega-oxidation is induced and/or upregulated by administering a cytochrome P450 enzyme or a functional part, derivative, and/or analogue, thereof. As stated before, omega-oxidation is catalyzed by at least one cytochrome P450 enzyme. Hence, omega-oxidation of a given fatty acid is induced and/or enhanced by administration of at least one cytP450 capable of catalyzing omega-oxidation of the given fatty acid, or a functional part, derivative, and/or analogue of the cytP450. Catalyzing omega-oxidation of a fatty acid means that at least one step of an omega-oxidation reaction is catalyzed. Preferably, hydroxylation of the carbon atom at the omega-end of a fatty acid is catalyzed. The terms “omega-oxidation catalyzing property,” “omega-hydroxylation capacity,” and “fatty acid hydroxylation capacity” are used herein interchangeably. In a preferred embodiment, a cytochrome P450 enzyme derived from the same species as the subject to be treated is used. For instance, in order to treat a human individual a human cytochrome P450 or a functional part, derivative, and/or analogue, thereof, is, preferably, used. Such human cytochrome P450 molecule is, for instance, produced by an expression system that has been provided with a nucleic acid sequence encoding a human cytochrome P450 enzyme or a functional part, derivative, and/or analogue, thereof.

In another embodiment, omega-oxidation is induced and/or upregulated by administration of a nucleic acid sequence encoding a cytochrome P450 enzyme or a functional part, derivative, and/or analogue of the enzyme. According to this embodiment, a subject suffering from, or at risk of suffering from, a disease involving accumulation of a given fatty acid is provided with a nucleic acid sequence encoding a cytP450 capable of catalyzing omega-oxidation of the fatty acid. Uses of nucleic acid sequences for administration of a proteinaceous molecule are well known in the art and need no further description herein. Gene delivery vehicles for introducing a nucleic acid sequence into an organism are well known in the art. For instance, a (retro) viral vector is used. The nucleic acid is either constitutively or inducibly expressed. In one embodiment, expression of the nucleic acid is controlled by an inducible promoter. In one embodiment, the nucleic acid sequence is only significantly expressed in one, or several, kind(s) of tissue(s). For instance, the nucleic acid sequence is operably linked to a promoter, which is specific for one, or several, kind(s) of tissue(s).

It is, of course, also possible to use other kinds of nucleic acid structures such as, but not limited to, a DNA/RNA helix, peptide nucleic acid (PNA), locked nucleic acid (LNA), and/or a ribozyme.

A functional part of a cytochrome P450 enzyme is defined as a part of the enzyme which has, at least, one same enzymatic property in kind, not necessarily in amount. By an enzymatic property is, preferably, meant the capability to catalyze omega-oxidation of a given fatty acid. A functional derivative of a cytP450 is defined as a cytP450 which has been altered such that at least one enzymatic property of the molecule is essentially the same in kind, not necessarily, in amount. A derivative can be provided in many ways, for instance, through conservative amino acid substitution. Conservative amino acid substitution involves replacement of an amino acid with another amino acid with generally similar properties (size, hydrophobicity, etc.), such that the overall functioning is not seriously affected.

A person skilled in the art is well able to generate analogous compounds of a cytP450. This can, for instance, be done through screening of a peptide library. Such an analogue has essentially the same immunogenic properties of the protein in kind, not necessarily in amount. An analogue of a cytP450, for instance, comprises a fusion protein.

A method or use of the invention, preferably, involves upregulation of the availability, preferably, the amount and/or omega-hydroxylation capacity, of at least one endogenous cytochrome P450 enzyme. An advantage of upregulating an organism's own pathway is that possible adverse side effects caused by exogenous enzymes are, at least, in part, avoided.

In one preferred embodiment, omega-oxidation of a given fatty acid is induced and/or enhanced by a compound capable of increasing the availability of at least one cytochrome P450 enzyme capable of catalyzing omega-oxidation of the fatty acid. The availability of cytP450, for instance, comprises the amount of cytP450 that is capable of catalyzing omega-oxidation, and/or the omega-hydroxylation capacity of a cytP450 enzyme. In a preferred embodiment, a compound capable of increasing the amount of at least one cytochrome P450 enzyme is used. In other embodiments, the availability of a cytochrome P450 enzyme is increased by removing a compound bound to the cytP450 by transport of the cytP450 to a desired site, et cetera. Additionally, or alternatively, a compound is used that is capable of enhancing the omega-hydroxylation capacity of a cytP450. By enhancing the omega-hydroxylation capacity of a cytochrome P450 enzyme is meant that at least one parameter of an omega-hydroxylation reaction catalyzed by the cytochrome P450 enzyme is improved. For instance, in one embodiment, a compound is used which enhances the affinity with which cytP450 is capable of binding a given fatty acid. In another embodiment, a compound is used which enhances the reaction rate of omega-hydroxylation catalyzed by a cytP450. Various other ways of enhancing the omega-hydroxylation capacity of a cytochrome P450 enzyme are known in the art.

An amount of a cytochrome P450 enzyme is upregulated in various ways. In one embodiment, degradation of a cytochrome P450 enzyme is, at least, in part, inhibited. This results in a higher amount of cytochrome P450 in a cell. In one preferred embodiment, an amount of a cytP450 is upregulated by inducing and/or enhancing expression of the cytP450. This is, for instance, performed by administration of a compound capable of inducing or enhancing an inducible cytP450-specific promoter. Many cytP450 enzyme genes are under control of at least one inducible promoter. In that case, it is possible to induce and/or enhance at least one inducible cytP450-specific promoter. This is, for instance, performed by administration of one or more compounds capable of inducing and/or enhancing at least one promoter. In one preferred embodiment, at least two inducible cytP450-specific promoters are induced or enhanced. In one embodiment, a compound capable of inducing and/or enhancing a first cytP450-specific promoter and another compound capable of inducing and/or enhancing a second cytP450-specific promoter is used. Alternatively, or additionally, a compound capable of inducing at least two cytP450-specific promoters is used. In one embodiment, one or several compounds capable of inducing and/or enhancing promoters of at least two different cytP450 enzymes are used.

In one embodiment, the promoter is directly induced or enhanced by the compound. For instance, a compound capable of directly binding the promoter, thereby, inducing expression of cytP450, is suitable. Additionally, or alternatively, the promoter is indirectly induced or enhanced. In one preferred embodiment, a compound is used that is capable of binding another compound or complex, the resulting complex being capable of binding and/or inducing the cytP450 promoter. In one embodiment, a compound is used that is capable of binding a cytP450 enhancer or silencer.

In a preferred embodiment, expression of cytP450 is induced and/or enhanced by a ligand of a member of the nuclear hormone family. The nuclear hormone family is well known in the art and represents a group of transcription factors with a similar structure which are activated by different ligands. PPAR-alpha, LXR, FXR, PXR, and CAR are preferred examples of members of the nuclear hormone family. The nuclear hormone family member, preferably, comprises a compound which is naturally bound to retinoic X receptor (RXR), since RXR-nuclear hormone family—ligand complexes are particularly suitable for inducing and/or enhancing cytP450 expression by binding to a response element in the promoter region of a cytP450 gene. Preferably, a ligand of a peroxisome proliferator-activated receptor alpha (PPAR-alpha), liver X receptor (LXR), farnesoid X receptor (FXR), pregnane X receptor (PXR), or constitutively active receptor (CAR) is used. Suitable ligands of nuclear hormone family members are listed in Table 1 (derived from Honkakoski et al., 2000).

According to one embodiment, binding of a ligand to a member of a nuclear hormone family bound to RXR is followed by binding of the resulting RXR—nuclear hormone family member—ligand complex to an inducible promoter of a cytP450. The binding results in (enhanced) expression of cytP450. One embodiment, therefore, provides a method or use according to the invention, wherein the compound capable of inducing and/or upregulating omega oxidation comprises a compound capable of directly or indirectly inducing or enhancing an inducible promoter of cytP450. The compound, preferably, comprises a ligand of the nuclear hormone family, most preferably, a ligand of PPAR-alpha, LXR, FXR, PXR, and/or CAR.

A preferred example of suitable ligands of PPAR-alpha is fibrates. Fibrates are a class of amphipathic carboxylic acids. Fibrates are already used in the art for a range of metabolic disorders, mainly in order to reduce cholesterol and triglyceride levels in the blood. A use of a fibrate for inducing and/or upregulating omega-oxidation of a fatty acid is not shown nor suggested before. The use of a fibrate in a method of the present invention is preferred because fibrates are already known to be suitable for use as medicaments without—or with acceptable—side effects. In a preferred embodiment, bezafibrate, fenofibrate, gemfibrozil, and/or ciprofibrate are used. These fibrates are already used as a therapeutic agent in human beings, although for entirely different purposes. These fibrates are, thus, already known to be suitable for therapeutic use in humans, without—or with acceptable—side effects. In another preferred embodiment, rifampicin and/or phenyloin are used in order to at least partly counteract a disease involving accumulation of fatty acid. Like fibrates, these compounds are known therapeutics, albeit for different diseases. Administration of these compounds to human beings has already been approved. A use of rifampicin and/or phenyloin for inducing and/or upregulating omega-oxidation of a fatty acid is neither shown nor suggested before.

The invention, thus, provides a method or use of the invention wherein the compound capable of inducing and/or upregulating omega-oxidation of fatty acid comprises a fibrate, rifampicin, and/or phenyloin. In a preferred embodiment, the fibrate comprises bezafibrate, fenofibrate, gemfibrozil, and/or ciprofibrate. In a further preferred embodiment, the compound comprises a compound as listed in Table 1. Of course, various other compounds capable of directly or indirectly inducing or enhancing the availability of a cytP450 enzyme are suitable for use in a method of the invention.

The art also provides various methods for enhancing the omega-hydroxylation capacity of a cytochrome P450 enzyme. For instance, a compound capable of enhancing enzymatic activity is administered. Alternatively, or additionally, a compound capable of increasing the availability of a substrate of cytP450 is used. For instance, a compound capable of increasing solubilization of fatty acid is used. In one embodiment, a compound is used which enhances binding of a fatty acid to a cytP450 enzyme.

In one aspect, a method or use of the invention is provided wherein the cytochrome P450 enzyme comprises a human cytochrome P450 enzyme. Inducing and/or upregulating the availability of at least one human cytochrome P450 enzyme is particularly suitable for therapeutic treatment of a human individual. Preferably, the availability of endogenous human cytP450 is enhanced.

In one embodiment, a method or use of the invention is combined with a conventional treatment method, such as enzyme replacement therapy. Additionally, or alternatively, at least two embodiments of a method of the present invention are performed. For instance, a method comprising upregulating and/or enhancing a cytochrome P450-specific promoter in an individual is combined with administration of cytochrome P450 to the individual. The at least two embodiments may be performed simultaneously. It is also possible to first start with one method of the invention, and, subsequently, start with another method of the invention. In one embodiment, a compound capable of upregulating and/or enhancing a cytochrome P450-specific promoter such as a ligand of a nuclear hormone family member is administered together with (at least one nucleic acid encoding) at least one cytP450. In another embodiment, the compound capable of upregulating and/or enhancing a cytochrome P450-specific promoter and the (at least one nucleic acid encoding) at least one cytP450 are administered at different time points.

The invention, furthermore, provides a use of a non-human animal suffering from, or at risk of suffering from, a disease involving accumulation of a fatty acid for determining whether a compound is capable of inducing and/or upregulating omega-oxidation of the fatty acid. It is, for instance, tested whether administration of a candidate compound to such animal results in decreased accumulation of fatty acid. Decreased fatty acid accumulation is, for instance, demonstrated by measuring the fatty acid(s) in plasma and/or tissues taken from treated and non-treated animals using, for instance, gaschromatography and/or tandem mass spectrometry. If accumulation of the fatty acid is decreased, it indicates that the candidate compound is capable of at least in part inducing and/or enhancing omega-oxidation of the fatty acid.

Moreover, such animal is suitable for testing possible negative side effects of a compound, which is capable of inducing and/or upregulating omega-oxidation of a fatty acid.

The invention, therefore, provides a method for determining whether and/or to what extent a compound is capable of inducing and/or upregulating omega-oxidation of a fatty acid, comprising providing a non-human animal suffering from, or at risk of suffering from, a disease involving accumulation of the fatty acid with the fatty acid and with the compound, determining the extent of accumulation of the fatty acid in the animal, and comparing the extent of accumulation with the extent of accumulation of the fatty acid in the same kind of non-human animal which is not, or to a significantly lesser extent, provided with the compound.

In one embodiment, the fatty acid comprises C26:O or phytanic acid.

### SUMMARY OF THE INVENTION

Two non-limiting examples of fatty acid accumulation disorders that are at least, in part, counteracted with a method or use of the present invention are Refsum disease and X-linked adrenoleukodystrophy.

Adult Refsum Disease (ARD) is an autosomal recessive disorder caused by deficient alpha-oxidation of phytanic acid (3,7,11,15-tetramethylhexadecanoic acid). In the majority of patients this is due to mutations in the gene encoding phytanoyl-CoA hydroxylase, a peroxisomal enzyme. In a subset of patients, mutations in the PEX7 gene have been found. Phytanoyl-CoA hydroxylase catalyzes the first step in the alpha-oxidation pathway of 3-methyl branched-chain fatty acids. These fatty acids require alpha-oxidation for their degradation since the 3-methyl group blocks breakdown by regular beta-oxidation. During alpha-oxidation, phytanic acid is converted into its n−1 analogue pristanic acid (2,6,10,14-tetramethylpentadecanoic acid), which can readily be degraded by peroxisomal beta-oxidation.

The deficiency of alpha-oxidation in ARD patients leads to the gradual accumulation of phytanic acid. Elevated phytanic acid in the absence of abnormalities in any of the other peroxisomal parameters, including plasma, very long chain fatty acids, bile acid intermediates, and erythrocyte plasmalogens is suggestive for ARD. Classical symptoms are: progressive retinitis pigmentosa, peripheral neuropathy, anosmia, and cerebellar ataxia. The only treatment available, at the moment, is a diet low in phytanic acid, which may be preceded by plasmapheresis. This alleviates the phytanic acid accumulation and slows down the progression of the disease.

X-linked adrenoleukodystrophy (X-ALD) is an inherited metabolic disorder involving accumulation of C26:O. People with ALD accumulate high levels of saturated, very long chain fatty acids in their brain and adrenal cortex because the fatty acids cannot be broken down. X-ALD is clinically heterogeneous with at least 6 subtypes, of which childhood cerebral ALD (CCALD) and adrenomyeloneuropathy (AMN) are most frequent occurring in >80% of patients.

In one embodiment, a method or use of the invention in order to at least, in part, counteract accumulation of phytanic acid and/or C26:O is provided. According to the present invention, the human cytochrome P450 enzymes CYP4A11, CYP4F2, CYP4F3A, and CYP4F3B are capable of catalyzing omega-oxidation of phytanic acid. Omega-oxidation of phytanic acid leads to 1,16-phytanedioic acid, which is further degraded by beta-oxidation from its omega-end. Hence, increasing the availability of at least one of these human cytochrome P450 enzymes results in, at least, partial treatment of Refsum disease. In one embodiment, Refsum disease is, therefore, at least, in part, counteracted by administration of CYP4A11, CYP4F2, CYP4F3A, and/or CYP4F3B, or a functional part, derivative, and/or analogue, thereof. The enzymes are suitable for use as a medicament. The invention, therefore, provides CYP4A11, CYP4F2, CYP4F3A, and/or CYP4F3B, for use as a medicament. A use of CYP4A11, CYP4F2, CYP4F3A, and/or CYP4F3B, or a functional part, derivative, and/or analogue, thereof, for the preparation of a medicament for, at least, in part, treating Refsum disease is, therefore, also provided herewith.

Another embodiment provides a method or use of the invention wherein a compound is used which compound, or its metabolite, is capable of inducing or upregulating the availability, preferably, the amount and/or the omega-hydroxylation capacity, of CYP4A11, CYP4F2, CYP4F3A, and/or CYP4F3B. Such compound is also suitable for the preparation of a medicament. A use of a compound, which is capable of increasing the amount and/or the omega-hydroxylation capacity of CYP4A11, CYP4F2, CYP4F3A, and/or CYP4F3B, or whose metabolite is capable of increasing the amount and/or the omega-hydroxylation capacity of CYP4A11, CYP4F2, CYP4F3A, and/or CYP4F3B, for the preparation of a medicament for, at least, in part, treating Refsum disease is, therefore, also provided herewith.

Furthermore, the invention provides a method for, at least, in part, treating Refsum disease the method comprising administering a compound, which is capable of increasing the amount and/or the omega-hydroxylation capacity of CYP4A11, CYP4F2, CYP4F3A, and/or CYP4F3B, or whose metabolite is capable of increasing the amount and/or the omega-hydroxylation capacity of CYP4A11, CYP4F2, CYP4F3A, and/or CYP4F3B, to a subject suffering from, or at risk of suffering from, Refsum disease.

According to another aspect of the invention, the human cytochrome P450 enzymes CYP4F2 and/or CYP4F3B are capable of catalyzing omega-oxidation of C26:O. Hence, increasing the availability of CYP4F2 and/or CYP4F3B results in, at least, partial treatment of X-linked adrenoleukodystrophy. In one embodiment, X-linked adrenoleukodystrophy is, therefore, at least, in part, counteracted by administration of CYP4F2 and/or CYP4F3B, or a functional part, derivative, and/or analogue, thereof. CYP4F2 and/or CYP4F3B, or a functional part, derivative, and/or analogue, thereof, for use as a medicament is also provided herewith, as well as a use of CYP4F2, CYP4F3B, or a functional part, derivative, and/or analogue, thereof, for the preparation of a medicament for, at least, in part, treating X-linked adrenoleukodystrophy.

One embodiment provides a method or use of the invention, wherein a compound is used, which compound, or whose metabolite, is capable of inducing or upregulating the availability, preferably, the amount and/or the omega-hydroxylation capacity, of CYP4F2 and/or CYP4F3B. Such compound is also suitable for the preparation of a medicament. The invention, therefore, also provides a use of a compound, which is capable of increasing the amount and/or the omega-hydroxylation capacity of CYP4F2 and/or CYP4F3B, or whose metabolite is capable of increasing the amount and/or the omega-hydroxylation capacity of CYP4F2 and/or CYP4F3B, for the preparation of a medicament for, at least, in part, treating X-linked adrenoleukodystrophy. A method, for at least, in part, treating X-linked adrenoleukodystrophy the method comprising administering a compound, which is capable of increasing the amount and/or the omega-hydroxylation capacity of CYP4F2 and/or CYP4F3B, or whose metabolite is capable of increasing the amount and/or the omega-hydroxylation capacity of CYP4F2 and/or CYP4F3B, to a subject suffering from, or at risk of suffering from, X-linked adrenoleukodystrophy is also provided herewith.

The invention, furthermore, provides a kit comprising a compound capable of increasing the amount and/or the omega-hydroxylation capacity of an enzyme selected from the group consisting of CYP4A11, CYP4F2, CYP4F3A, and/or CYP4F3B, or whose metabolite is capable of increasing the amount and/or the omega-hydroxylation capacity of an enzyme selected from the group consisting of CYP4A11, CYP4F2, CYP4F3A, and/or CYP4F3B. The kit is particularly suitable for, at least, in part, counteracting Refsum disease and/or X-linked adrenoleukodystrophy. In one embodiment, at least two compounds capable of increasing the amount and/or the omega-hydroxylation capacity of an enzyme selected from the group consisting of CYP4A11, CYP4F2, CYP4F3A, and/or CYP4F3B are used. One embodiment, therefore, provides a kit comprising a first compound capable of increasing the amount and/or the omega-hydroxylation capacity of a first enzyme selected from the group consisting of CYP4A11, CYP4F2, CYP4F3A, and/or CYP4F3B, or whose metabolite is capable of increasing the amount and/or the omega-hydroxylation capacity of a first enzyme selected from the group consisting of CYP4A11, CYP4F2, CYP4F3A, and/or CYP4F3B, and a second compound capable of increasing the amount and/or the omega-hydroxylation capacity of a second enzyme selected from the group consisting of CYP4A11, CYP4F2, CYP4F3A, and/or CYP4F3B, or whose metabolite is capable of increasing the amount and/or the omega-hydroxylation capacity of a second enzyme selected from the group consisting of CYP4A11, CYP4F2, CYP4F3A, and/or CYP4F3B.

The first and second compounds are, preferably, different from each other. In one embodiment, the first and second enzymes are different enzymes.

The invention, furthermore, provides a kit dedicated to the treatment of X-linked adrenoleukodystrophy. The kit, preferably, comprises a compound capable of increasing the availability, preferably, the amount and/or the omega-hydroxylation capacity, of CYP4F2 and/or CYP4F3B, since the human cytochrome P450 enzymes CYP4F2 and/or CYP4F3B are capable of catalyzing omega-oxidation of C26:O, resulting in, at least, partial treatment of X-linked adrenoleukodystrophy. In one preferred embodiment, the availability of CYP4F2 and CYP4F3B is enhanced. One embodiment, therefore, provides a kit comprising a compound capable of increasing the amount and/or the omega-hydroxylation capacity of CYP4F2, or whose metabolite is capable of increasing the amount and/or the omega-hydroxylation capacity of CYP4F2, and a compound capable of increasing the amount and/or the omega-hydroxylation capacity of CYP4F3B, or whose metabolite is capable of increasing the amount and/or the omega-hydroxylation capacity of CYP4F3B. The two compounds are, preferably, different from each other.

The invention is further explained in the following examples. These examples do not limit the scope of the invention, but merely serve to clarify the invention. Many alternative embodiments can be carried out, which are within the scope of the present invention.

### EXAMPLES

### Example 1

Material and Methods

Materials

Phytanic acid and 3-hydroxyheptadecanoic acid were purchased from Larodan Fine Chemicals AB (Malmo, Sweden). NADPH and NAD⁺ were obtained from Roche (Mannheim, Germany). Clotrimazole, ketoconazole, bifonozole and miconazole were purchased from Sigma (St. Louis, Mo.). Methyl-beta-cyclodextrine was from Fluka (Buchs, Switzerland).

Preparation of Rat Liver Microsomes

Microsomes were isolated from rat livers by differential centrifugation essentially as described Baudhuin et al. (Baudhuin, 1964). To this end, Male Wistar rats fed a standard laboratory diet, were fasted overnight before sacrifice and removal of the liver. The livers were rapidly chilled and washed several times in buffer containing 250 mM sucrose, 0.5 mM EDTA, 2 mM MOPS/KOH (final pH 7.4). Subsequently, the livers were minced and homogenized with a Potter S homogenizer (B. Braun, Germany) with a teflon pestle at 500 rpm (5 strokes), followed by centrifugation of the homogenate for 10 min at 550×g. The obtained post-nuclear supernatant was subjected to centrifugation at 22,500×g for 10 min to remove mitochondria and lysosomes. Finally, the microsomal fraction was obtained by centrifugation of the supernatant for 3 h at 32,000×g. The microsomal pellet fraction was taken up in PBS containing 5 mM DTT and divided into small aliquots, which were stored at −80° C. The microsomes were sonicated 3 times for 10 s at 8 Watt before each experiment. The protein concentration of the microsomal fraction was determined with the method described by Bradford (Bradford, 1976).

Phytanic Acid omega(-1)-hydroxylase Assay

The standard reaction mixture consisted of 100 mM potassium phosphate buffer pH 7.4 and rat liver microsomes (1 mg/ml end concentration) plus phytanic acid dissolved in DMSO (200 μM end concentration, unless indicated otherwise). Reactions were initiated by addition of NADPH at a final concentration of 1 mM. The final reaction volume was 0.2 ml. Reactions were terminated by addition of 0.2 ml 1 M HCl. Subsequently, 1 ml PBS was added followed by addition of 0.1 ml 12.1 M HCl. The internal standard (IS, 10 nmol 3-hydroxyheptadecanoic acid in 20 μl ethanol) was added to this aqueous mixture. The samples were extracted twice with 6 ml ethylacetate-diethylether (1:1 v/v). The organic layer was collected and the solvents evaporated under vacuum using a rotary evaporator at room temperature. The residue was dissolved in 4 ml ethylacetate and further dried with MgSO₄. After spinning down the MgSO₄, the solution was transferred to 4 ml reaction vials and the solvent evaporated under nitrogen. To enable gas chromatography-mass spectrometry (GS/MS) analysis the extracted fatty acids were derivatized to their corresponding trimethylsilyl (TMS) compounds essentially using the procedure described by Chalmers and Lawson (Chalmers, 1982). TMS ester/ether formation was performed with 40 μl N,O-bis-(trimethylsilyl)trifluoroacetamide (BSTFA) containing 1% trimethylchlorosilane (TMCS) and 10 μl pyridine. The vials were sealed with a Teflon-lined screwcap and incubated at 80° C. for 1 hour. After the incubation the solution could be directly used for GC-MS analysis.

GC/MS

GC/MS was performed on a Hewlett-Packard 6890 gas chromatograph coupled to a Hewlett-Packard 5973 mass-selective detector (Palo Alto, Calif.). Samples (1 μl) were injected in the splitless mode (Hewlett Packard 7683 injector) and analyzed on a CP-Sil 5 CB low bleed MS column (25 m×0.30 μm) (Chrompack, Middelburg, The Netherlands). The oven temperature was programmed as follows: 70° C. for 2 min, 5° C./min to 120° C., 7° C./min to 260° C., 3.5 min hold at 260° C. then 15° C./min to 275, hold for 10 min. The identity of the substrate, the internal standard, and the reaction product(s) was verified by taking mass spectra of the pertrimethylsilylated derivatives in the scanning electron impact mode. The single ion monitoring (SIM) mode was applied for the detection of the respective (M−15)⁺ ions (m/z 369 and 457; masses of the molecular ions minus one methyl group of the TMS derivatives of phytanic acid and omega(-1)-hydroxyphytanic acid respectively). Analyte quantification was done by integration of the peaks followed by dividing the analyte peak areas with the area of the internal standard (TMS derivative of 3-hydroxyheptadecanoic acid, monitored ion m/z 233).

Results

Hydroxylation of Phytanic Acid by Rat Liver Microsomes

In order to study the omega-oxidation of phytanic acid, rat liver microsomes were incubated in a phosphate buffered medium containing phytanic acid. When NADPH was added, two products appeared in the chromatogram, one with a retention time of 29.1 min and the other with a retention time of 29.7 min (FIG. 1). The major peak was identified as omega-hydroxyphytanic acid (16-hydroxyphytanic acid) according to its mass spectrum (FIG. 2A). Mass spectral analysis of this peak revealed the presence of a fragment (m/z=103) characteristic for omega-hydroxyacids, representing the terminal CH₂OSi(CH₃)₃₋ moiety. The minor peak corresponds to (omega-1)-hydroxyphytanic acid (15-hydroxyphytanic acid) (FIG. 2B), the product of (omega-1)-hydroxylation. A general characteristic of the TMS-derivatives of the hydroxyfatty acids is cleavage of the molecule adjacent to the hydroxyl group. It was deduced from the mass spectrum in FIG. 2B that the base peak at m/z=131 represents the (CH₃)₂COSi(CH₃)₃₋ moiety, in analogy with the mass spectrum of 3-hydroxyisovaleric acid.

Optimization of the Hydroxylase Assay

The hydroxylation assay was further optimized for the formation of omega-hydroxyphytanic acid. First the influence of methyl-beta-cyclodextrin, added to increase solubilization of the substrate, on the formation of omega-hydroxyphytanic acid was tested. FIG. 3A shows that methyl-beta-cyclodextrin has a positive effect on the assay with an optimum concentration of 0.75 mg/ml. Higher methyl-beta-cyclodextrin concentrations had a negative effect on the assay, presumably due to decreased substrate availability. To determine the optimal pH value for our assay a combined buffer system with 50 mM potassium phosphate/50 mM pyrophosphate was used to cover the pH range of 6.6 to 9.1. The result depicted in FIG. 3B shows an optimum pH of 7.6. Accordingly, all subsequent experiments were performed in 0.1 M potassium phosphate at pH 7.6.

Our next aim was to analyze the kinetics of the hydroxylation of phytanic acid under the conditions determined in the previous experiments. I already established that NADPH was an essential component of the reaction mixture (FIG. 1). The NADPH dependency of the reaction was studied in more detail by performing the assay at different NADPH concentrations. For this purpose, I included a NADPH regenerating system (10 mM isocitrate, 10 mM MgCl₂ and 0.08 U isocitrate dehydrogenase) in the assay mixture since large amounts of NADPH were consumed during the assay (data not shown). The formation of omega-hydroxyphytanic acid followed simple Michaelis Menten kinetics (FIG. 3C). The apparent Kₘ for NADPH derived from the Lineweaver-Burke plot (insert in FIG. 3C) was 35 μM.

Subsequently, I determined the effect of increasing phytanic acid concentrations on the formation of omega-hydroxyphytanic acid. To this end, different concentrations of phytanic acid were added in a fixed molar ratio between phytanic acid and methyl-beta-cyclodextrin (FIG. 3D). An apparent Km of 114±9 μM was found.

Based on the experiments described above, I selected the following assay conditions: 0,75 mg/ml methyl-beta-cyclodextrin, 100 mM potassium phosphate (pH 7.6), 1 mM NADPH, and 200 μM phytanic acid. Under these conditions, formation of omega-hydroxyphytanic acid was linear with time up to 60 min, and with protein up to 1 mg/ml (data not shown).

Effect of Imidazole Derivatives on the Formation of Omega-Hydroxyphytanic Acid

Imidazole antimycotics are known inhibitors of cytochrome P450 enzymes (Halpert, 1995; Zhang, 2002; Maurice, 1992). To measure the influence of four different imidazole derivatives on the formation of omega-hydroxyphytanic acid, I studied the effect of different concentrations of these compounds on the formation of omega-hydroxyphytanic acid (FIG. 4A) and (omega-1)-hydroxyphytanic acid (FIG. 4B). FIG. 4A shows that omega-hydroxyphytanic acid formation was inhibited by all four compounds with bifonazole as the most potent inhibitor, followed by ketoconazole, miconazole, and clotrimazole. Interestingly, a different picture was observed if the effect of the four imidazole derivatives was studied on the formation of the (omega-1)-compound with miconazole as most potent inhibitor, followed by ketoconazole and bifonazole. Remarkably, clotrimazole showed a stimulatory effect at low concentrations with little inhibition at the highest concentrations used (100 μM).

To summarize, phytanic acid is hydroxylated to its omega and omega-1 hydroxy analogues in rat liver microsomes. The enzyme(s) responsible for phytanic acid omega- and (omega-1)-hydroxylation were shown to be NADPH dependent. Moreover, the formation of the omega- and omega-1 hydroxy analogues of phytanic acid was inhibited by imidazole antimycotics. The inhibition by the imidazole derivatives showed a different pattern for the two products. Hence, this strongly suggests that different members of the cytochrome P450 multi-enzyme family are responsible for the formation of omega- and (omega-1)-phytanic acid.

### Example 2

In Example 1, I have shown that rat liver microsomes are able to omega-hydroxylate phytanic acid. This reaction is catalyzed by at least one member of the cytochrome P450 enzyme family and results in the formation of two metabolites, omega- and (omega-1)-hydroxyphytanic acid. Cytochrome P450 enzymes are readily inducible by a variety of drugs (Waxman, 1999; Honkakoski, 2000). According to the present invention, induction of at least one cytochrome P450 involved in phytanic acid omega-hydroxylation leads to an increased clearance of phytanic acid in Refsum patients with obvious implications for the treatment of these patients. In Example 1, I have extended our studies from rat liver microsomes to human liver microsomes.

Materials and Methods

Materials

Phytanic acid was obtained from the VU University Medical Center Metabolic Laboratory (Dr. H. J ten Brink, Amsterdam, the Netherlands). 3-Hydroxyheptadecanoic acid was from Larodan Fine Chemicals AB (Malmö, Sweden). NADPH and NAD⁺ were obtained from Roche (Mannheim, Germany). Clotrimazole, ketoconazole, bifonozole and miconazole were obtained from Sigma (St. Louis, Mo., USA). Methyl-beta-cyclodextrine was from Fluka (Buchs, Switzerland). N,O-bis-(trimethylsilyl)trifluoroacetamide (BSTFA) containing 1% trimethylchlorosilane (TMCS) was from Pierce (Rockford, Ill., USA). Pooled human liver microsomes were obtained from BD Gentest™ (Woburn, Mass., USA). Rat liver microsomes were prepared from Male Wistar rats by differential centrifugation as described in detail in Example 1. Other chemicals used were of the highest quality possible.

Phytanic Acid omega- and omega-1-hydroxylation Assay

Essentially, the same conditions were used as described in Example 1, except for the final reaction volume which was 0.1 ml. In brief, phytanic acid dissolved in DMSO was added to a solution of microsomes (1 mg/ml final concentration) in 100 mM potassium phosphate buffer (pH 7.7), containing phytanic acid at a final concentration of 200 μM, unless indicated otherwise. Reactions were initiated by adding NADPH (final concentration 1 mM) and terminated by addition of 0.1 ml 1 M HCl. Subsequently, 0.5 ml phosphate-buffered saline (PBS) was added followed by 50 μL 12.1 M HCl. After addition of the internal standard (IS, 2 nmol 3-hydroxyheptadecanoic acid in 20 μl ethanol) the samples were extracted twice with 6 ml ethylacetate-diethylether (1:1 v/v). The organic layers were collected and the solvents evaporated. The residue was dissolved in 4 ml ethylacetate and further dried with anhydrous MgSO₄ and again evaporated. To enable gas chromatography-mass spectrometry (GS/MS) analysis the extracted fatty acids were derivatized to their corresponding trimethylsilyl (TMS) compounds essentially using the procedure described by Chalmers and Lawson (Chalmers, 1982). TMS ester/ether formation was performed by incubating the samples with 40 μl BSTFA containing 1% TMCS and 10 μl pyridine at 80° C. for 1 hour. After the incubation the solution was directly used for GC-MS analysis.

GC/MS

GC/MS was performed on a Hewlett-Packard 6890 gas chromatograph coupled to a Hewlett-Packard 5973 mass-selective detector (Palo Alto, Calif.). Samples (1 μl) were injected in the splitless mode (Hewlett-Packard 7683 injector) and analyzed on a CP-Sil 5 CB low bleed MS column (25 m×0.30 μm) (Chrompack, Middelburg, The Netherlands). The oven temperature program used is the same as described in Example 1. The single ion monitoring (SIM) mode was applied for the detection of the respective (M−15)⁺ ions (m/z 369 and 457; masses of the molecular ions minus one methyl group of the TMS derivatives of phytanic acid and omega(-1)-hydroxyphytanic acid, respectively). Analyte quantification was done by integration of the peaks followed by dividing the analyte peak areas by the area of the internal standard (TMS derivative of 3-hydroxyheptadecanoic acid, monitored ion m/z 233).

Results

Phytanic Acid Omega-Hydroxylation in Pooled Human Liver Microsomes

Our studies show that phytanic acid undergoes NADPH-dependent omega- and (omega-1)-hydroxylation in rat liver microsomes (see Example 1). To test whether this also occurs in human liver microsomes, the same assay was performed under the optimum conditions described for rat liver microsomes (see Materials and Methods section of Example 1). Under these conditions, the human liver microsomes, indeed, showed the capacity to produce omega- and (omega-1)-hydroxyphytanic acid (FIG. 5). The identity of the omega- and (omega-1)-hydroxylated products was confirmed by their corresponding fragmentation patterns as was done earlier when using rat liver microsomes (see Example 1). The major difference between rat and human microsomal systems is the ratio between the two products formed, as can be seen in FIG. 5. In pooled human microsomes the ratio omega: (omega-1) was 15.4±0.7, whereas, in rat liver microsomes this ratio was found to be 2.2±0.2 as measured in four separate experiments.

Optimization of the Phytanic Acid omega(-1)-hydroxylase Assay

The marked difference between the ratios of product formation between human and rat liver microsomes (FIG. 5) led us to optimize the hydroxylation assay in human liver microsomes for omega-hydroxyphytanic acid formation. First, I tested the effect of methyl-beta-cyclodextrin, which was used in the assay to increase the solubilization of phytanic acid. As shown in FIG. 6A, methyl-beta-cyclodextrin had a positive influence on the formation of omega-hydroxyphytanic acid, up to approximately 1 mg/ml. A further increase of methyl-beta-cyclodextrin in the assay had a negative effect on the rate of product formation, presumably caused by a decrease in the availability of the substrate. The same phosphate-based buffer system as used in Example 1 on rat liver microsomes (50 mM potassium phosphate/50 mM pyrophosphate) was used for the determination of the optimum pH of the reaction. The pH optimum of the reaction was 7.7 (FIG. 6B). All subsequent experiments were done at this particular pH.

In order to determine the Km for NADPH, the NADPH concentration was varied in the assay in combination with the use of a NADPH-regenerating system (10 mM isocitrate, 10 mM MgCl₂ and 0.08 U isocitrate dehydrogenase). The reaction followed simple Michaelis Menten kinetics (FIG. 6C) and from the Lineweaver Burke plot (insert FIG. 6C) a Km of 2 μM (means of duplicate experiments) could be deduced. This Km is considerably lower than the Km determined in rat liver microsomes (35 μM) (see Example 1).

Subsequently, the Km of the enzyme for phytanic acid was determined. Different concentrations of phytanic acid were used with a fixed ratio between phytanic acid and methyl-beta-cyclodextrin. The v versus [S] plot did not follow Michaelis-Menten kinetics, so the calculation of the respective Km from the Lineweaver Burke plot could not be done. Consequently, I estimated the Km for phytanic acid as the substrate concentration that shows half maximal omega-hydroxylation activity. Based on the data in FIG. 6D, an apparent Km of 80 μM was determined (FIG. 6D). The nonlinear kinetics may, at least, in part, be caused by inefficient solubilization of phytanic acid, although beta-methylcyclodextrin was used in the assay. In vivo, this problem is, for instance, overcome by a carrier protein which provides the substrate to the P450 enzyme, similar to sterol carrier protein 2 (SCP2) acting as a carrier protein for phytanoyl-CoA during alpha-oxidation (Mukherji, 2002). Liver fatty acid binding protein (L-FABP) is a possible candidate to play such a role during omega-hydroxylation based on the notion that L-FABP has a high affinity for phytanic acid outside of the peroxisome. L-FABP is already known to be required for regular branched-chain fatty acid uptake and metabolism by having a role in cytoplasmic fatty acid transport.

Effect of Azole Antimycotics

The formation of omega-hydroxyphytanic acid was shown to be inhibited by imidazole derivatives in rat liver microsomes, indicating that the reaction is catalyzed by a member of the cytochrome P450 enzyme family. To establish whether omega-hydroxylation of phytanic acid is also catalyzed by a cytochrome P450 protein in human liver microsomes, I performed activity measurements in the presence of different concentrations of the imidazole derivatives bifonazole, clotrimazole, ketoconazole and miconazole, which were all found to inhibit product formation (FIG. 7A). Ketoconazole appeared to be the most potent inhibitor and not bifonazole which was most potent in rat liver microsomes (see Example 1). As shown in FIG. 7, the effect of the inhibitors on (omega-1)-hydroxyphytanic acid formation was much more pronounced as compared to the inhibitory effect on omega-hydroxyphytanic acid formation. This is also clear from the IC50 values in Table 1.

The results of Example 2 illustrate that human liver microsomes are able to omega-hydroxylate phytanic acid. The formation of the products omega- and (omega-1)-hydroxyphytanic acid is NADPH dependent and inhibited by imidazole antimycotics.

### Example 3

Specific cytochrome P450 enzymes were tested for their capability of catalyzing omega-hydroxylation of phytanic acid with the following experiment:

Supersomes, in which each of the individual cytP450s were expressed, were obtained from commercial sources (BD Gentest™ (Woburn, Mass.)). At the day of the experiment an aliquot was taken from each of the different supersome preparations, followed by incubation in a standard reaction medium, containing 100 mmol/L potassium phosphate pH 7.8, 1 mmol/L NADPH, 0.75 mg/milliliter methyl-beta-cyclodextrine, and 0.2 mmol/L phytanic acid. Reactions were allowed to proceed for 30 minutes after which the formation of omega-hydroxy phytanic acid was quantified by means of gas chromatography and mass spectrometry analysis.

### Example 4

### Production of Mutant Mice

Construction of the Targeting Vector

Genomic clones were isolated from a 129 SVJ mouse λ-FIX II genomic library (Stratagene) using complete mPhyH cDNA as a probe. Positive phages were further screened with various parts of mPhyH cDNA as probes and finally two positive phages were selected, one containing the 5′ end to exon 6-7 and one containing exon 6-7 to the 3′ end of the PhyH gene. The two selected phages were purified and, subsequently, DNA was isolated, digested with NotI and subcloned into a low copynumber plasmid, pBR-GEM11, which was kindly provided by H. ten Riele (The Netherlands Cancer Institute, Amsterdam, The Netherlands). From both constructs, C4 and C11, a restriction map was made and, with various parts of mPhyH cDNA which were used as probes, the orientation of part of the gene in both constructs was determined. Based on the restriction maps, a large fragment of approximately 6.5 kb and a small fragment of approximately 2 kb were chosen to generate a targeting vector. A 4.4 kb BamHI-SmaI fragment containing exon 7-9 and 3′ flanking sequence was subcloned from C11 and used to isolate a 1.8 kb HindIII/ClaI fragment (“short arm”) that was ligated into a pBluescript-SK⁻ based plasmid which was already carrying the hygromycin B resistance gene. This plasmid was then linearized with XhoI, end-filled, cut with HindIII and ligated into pBluescript-SK⁻ (opened with HindIII and SmaI). The short arm and hygromycin B resistance gene were liberated from pBluescript-SK⁻ by using HindIII and NotI and, subsequently, ligated into pBR-GEM11 and opened with the same restriction enzymes. A 7.8 kb EcoRI fragment containing the 5′ flanking sequence and exon 1-3 was subcloned from C4 and used to isolate a 6.7 kb EcoRI/XhoI fragment (“long arm”) that was ligated into TOPO. The long arm was cut out of TOPO by using NotI and XhoI and ligated into pBR-GEM11, containing the short arm and the resistance gene, which was opened with NotI and XhoI. For electroporation of ES cells, either BamHI linearized targeting vector was used or the HindIII fragment containing the complete short arm, hygromycin B resistance gene and 6.3 kb of the long arm.

Culturing and Electroporation of ES Cells

The IB10 subclone from the E14 ES cell line was obtained from The Netherlands Cancer Institute (Amsterdam, The Netherlands) and grown on irradiated murine embryonic fibroblast feeder cells in GMEM supplemented with 10% FCS, 2 mM L-glutamine, 1 mM sodium pyruvate, 1× nonessential amino acids, 0.1 mM β-mercaptoethanol and 1000 U/ml LIF in a 5% CO₂-humidified incubator (REF Robanus). 6×10⁶ IB10 ES cells were electroporated with 30 μgram targeting DNA in a total volume of 300 μl PBS using a single pulse from a gene pulser (Bio-Rad) at 0.8 kV and 3 μF. Selection with hygromycin (150 μg/ml) was started the day after the electroporation. During selection, the ES cells were cultured in 60% BRL-conditioned medium without feeder cells.

Screening of ES Cells

Targeted ES cells were screened by PCR for homologous recombination, using the primers 5′-CGCGAAGGGGCCACCAAAGAAC (SEQ ID NO:1) and 5′-TCCCCAGAAAGCAAGCAAAAGACT (SEQ ID NO:2) and an annealing temperature of 58° C. These primers correspond to hygromycin B resistance marker and the 3′ genomic flank outside of the construct, respectively. A fragment of the expected size of 2.0 kb was amplified in 12 out of 740 clones. PCR positive clones were also analyzed by southern blot for correct 5′ homologous recombination. Genomic DNA was digested overnight with EcoRV and BamHI and hybridization of the southern blot was carried out with an external 5′ probe (EcoRI-HincII fragment, containing exon 1). This 5′ external probe hybridizes to a 12.3 kb fragment from wild type. When one allele of the PhyH gene is replaced with the targeting vector sequences by homologous recombination, an additional fragment of 10 kb appears. Several ES clones which had undergone the correct homologous recombination were verified for the correct karyotype (> 12/15 metaphase chromosome spreads with 40 chromosomes).

Generation and Breeding of Mutant Mice

ES cells from three targeted clones were injected in C57BL/6 blastocysts, resulting in twelve chimeric mice obtained from two ES clones. Five chimeras, three derived from one ES clone and two from another, showed germ-line transmission and were bred to FVB females. The grey offspring was analyzed by PCR on genomic ear DNA. The forward primer for the wild-type allele was 5′-CCTCTCCAATCTTAGTCGGTCCTTTCT (SEQ ID NO:3). The forward primer for the targeted PhyH allele was located in the promoter in front of hygromycine: 5′-CCTACCGGTGGATGTGGAATGTGT (SEQ ID NO:4). One reverse primer was used with both forward primers: 5′-AGCCCCCTAGCGTTTCCTCTGTGA (SEQ ID NO:5) at an annealing temperature of 59° C. The PCR product obtained from the wild-type allele was 205 bp, from the targeted allele 262 bp. Mice carrying the mutant PhyH allele were crossed. All animal experiments were conducted under the approval of the animal care committee of the KNAW (Royal Dutch Academy of Arts and Sciences).

Animal Feeding Studies

Six male mice, 8-9 weeks old, 25-30 grams were used for the phytol diet study. They were divided into 2 groups. One group was fed with 0.25% (W/w) phytol rodent chow diet (Arie Blok Diervoeding, Woerden, The Netherlands) the other group was fed with the identical rodent chow diet but without phytol. Equal number of male wild-type and heterozygous littermates were used for the study. All animals received water and food ad libitum for the treatment period of two weeks. They were kept individually and food intake and body weight were monitored daily. At the end of the treatment, between 9 and 10 a.m., mice were sacrificed with CO₂ and tissues were dissected and immediately frozen in liquid nitrogen. For the isolation of blood samples to measure various parameters, an identical animal study was carried out with the exception that the mice had been fasting 4 hours before they were sacrificed.

### Example 5

The mice described above are fed with 0.25% (w/w) phytol rodent chow diet (Arie Blok Diervoeding, Woerden, The Netherlands). The mice are divided into two groups. One group additionally receives a candidate compound. The other group serves as a negative control. All animals receive water and food ad libitum for the treatment period. They are kept individually and food intake and body weight are monitored daily. At the end of the treatment the mice are sacrificed with CO₂ and tissues are dissected and immediately frozen in liquid nitrogen. It is, subsequently, determined whether less phytanic acid accumulation occurs in the mice which were provided with the candidate compound, as compared to the mice which were not provided with the candidate compound.

For the isolation of blood samples to measure various parameters, an identical animal study is carried out with the exception that the mice have been fasting 4 hours before they are sacrificed. Phytanic acid will be measured in plasma and tissues of mice, which were provided with the candidate compound, or not, by means of gas chromatography/mass spectrometry, and/or tandem mass spectrometry.

### BRIEF DESCRIPTION OF THE DRAWINGS

FIG. 1. GC-MS (single-ion monitoring mode) chromatograms of extracts from rat liver microsomes incubated with phytanic acid in the absence (A) or presence (B) of NADPH. Spectrum analysis was done as described in Materials and Methods of Example 1. Peaks corresponding to the (M−15)⁺ (m/z=457) of hydroxylated phytanic acid metabolites are labeled I and II. 3-Hydroxyheptadecanoic acid (ion m/z=233) was used as internal standard (IS).

FIG. 2. Mass spectra of peaks labeled I and II in FIG. 1B. Based on the fragmentation pattern shown in (A) and (B), peak I was identified as the trimethylsilyl (TMS) derivative of (omega-1)-hydroxyphytanic acid, whereas peak II was identified as the TMS-derivative of omega-hydroxyphytanic acid.

FIG. 3. Optimization of the phytanic acid hydroxylase activity in rat liver microsomes.

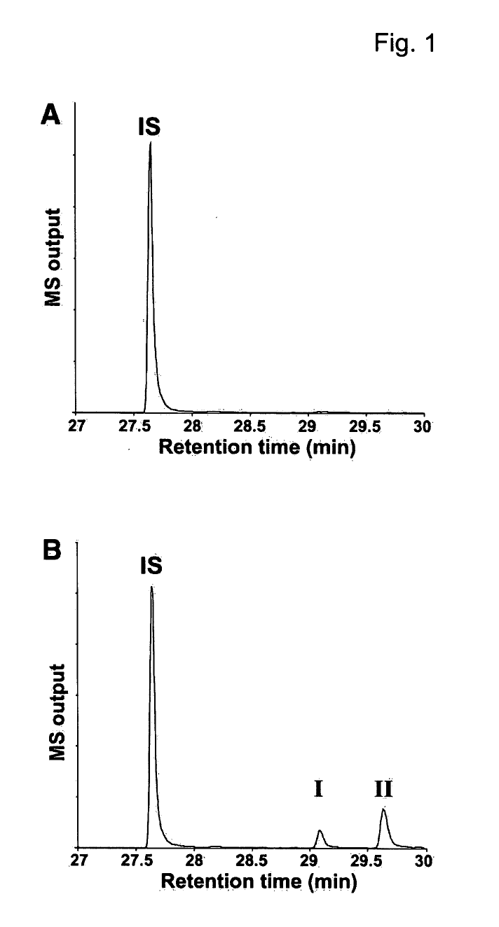

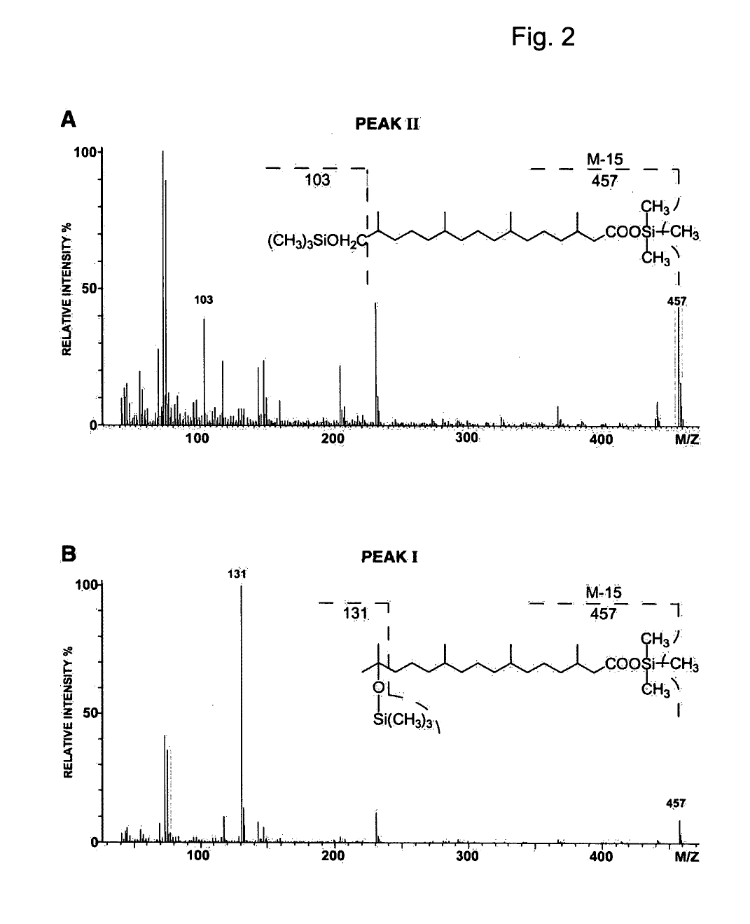

A: The effect of different concentrations of methyl-beta-cyclodextrin on the formation of omega-hydroxyphytanic acid (omega-HPA) in rat liver microsomes was determined under conditions described in Materials and Methods of Example 1.

B: The pH dependency of phytanic acid omega-hydroxylation. The hydroxylase assay was performed as in A, with the exception of the use of a combined buffer containing 50 mM potassium phosphate and 50 mM pyrophosphate and 0.75 mg/ml methyl-beta-cyclodextrin.

C: The effect of the NADPH concentration on phytanic acid omega-hydroxylation in the presence of a NADPH regenerating system (10 mM isocitrate, 10 mM MgCl₂ and 0.08 U isocitrate dehydrogenase). The experimental set-up was as described in Materials and Methods of Example 1, with the exception of using a 100 mM potassium phosphate buffer (pH 7.6) and 0.75 mg/ml methyl-beta-cyclodextrin. The Kₘ for NADPH was determined to be 35 μM as derived from the Lineweaver Burke plot (insert).

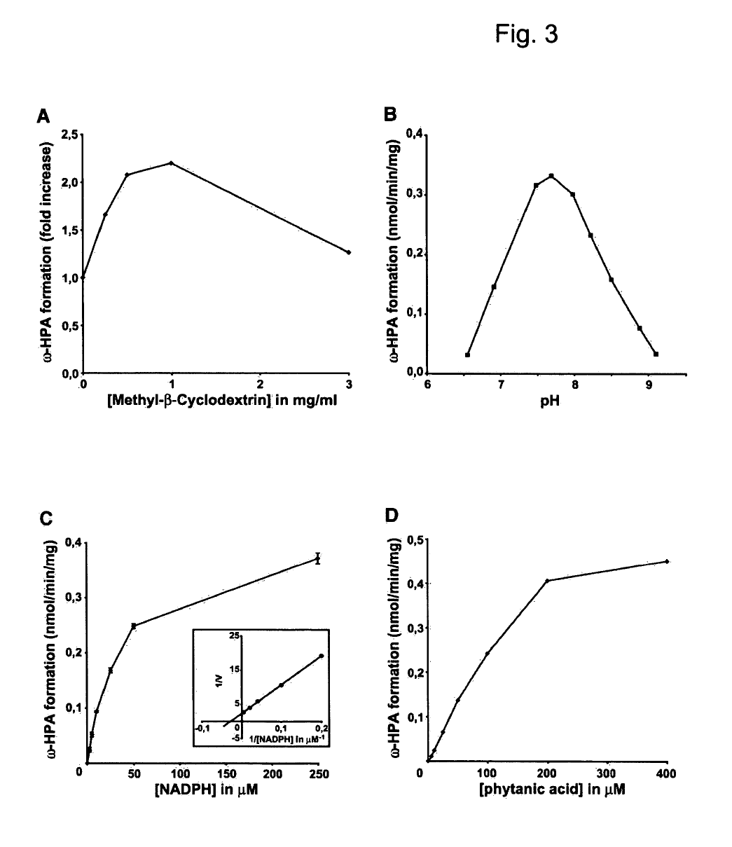

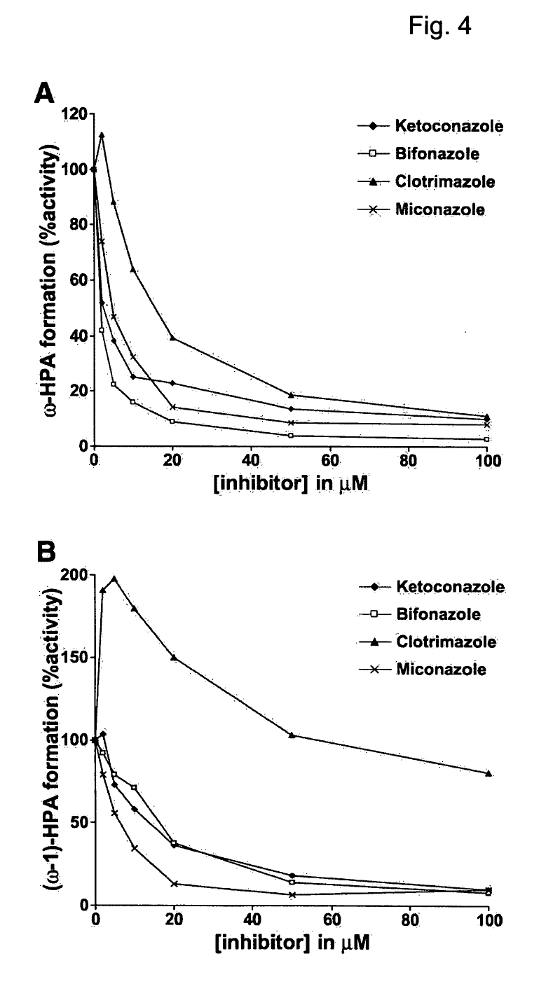

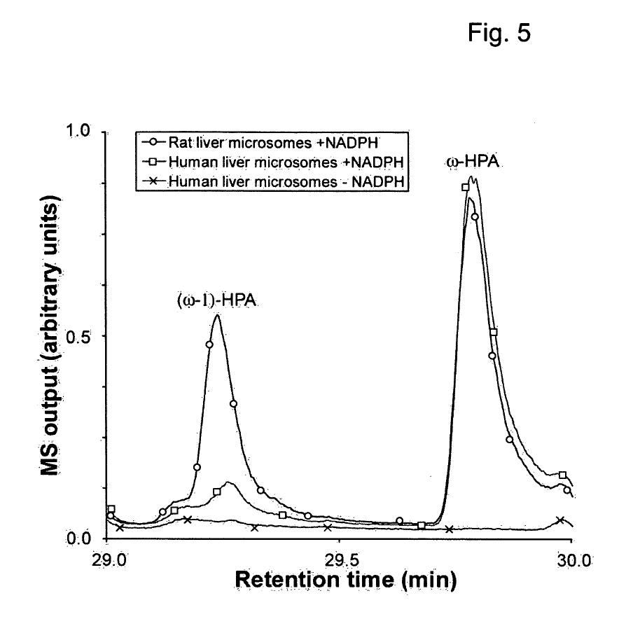

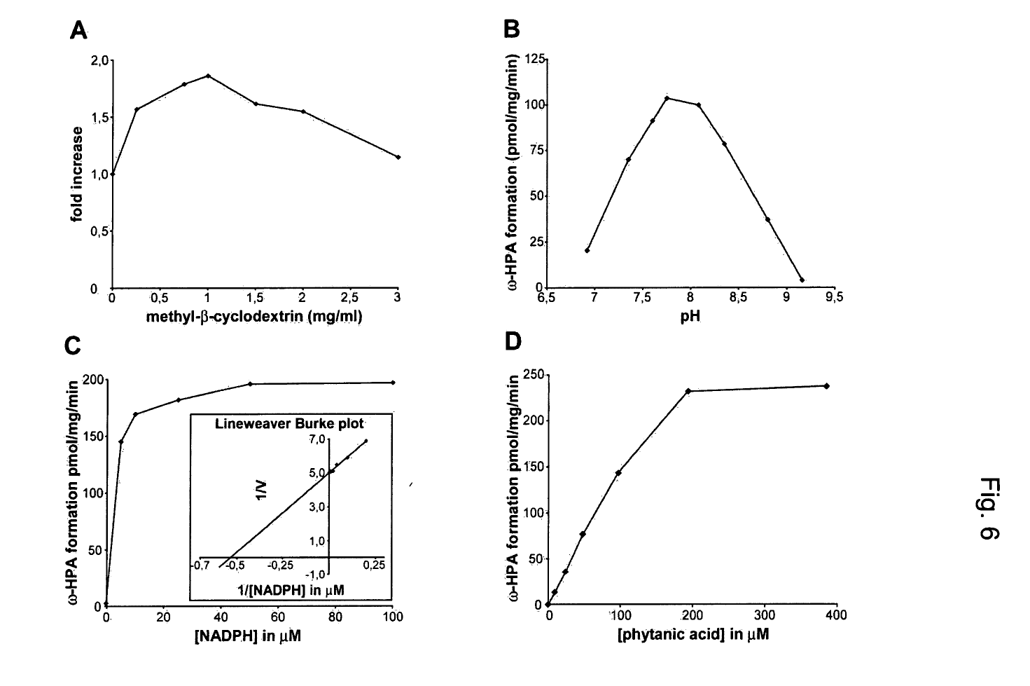

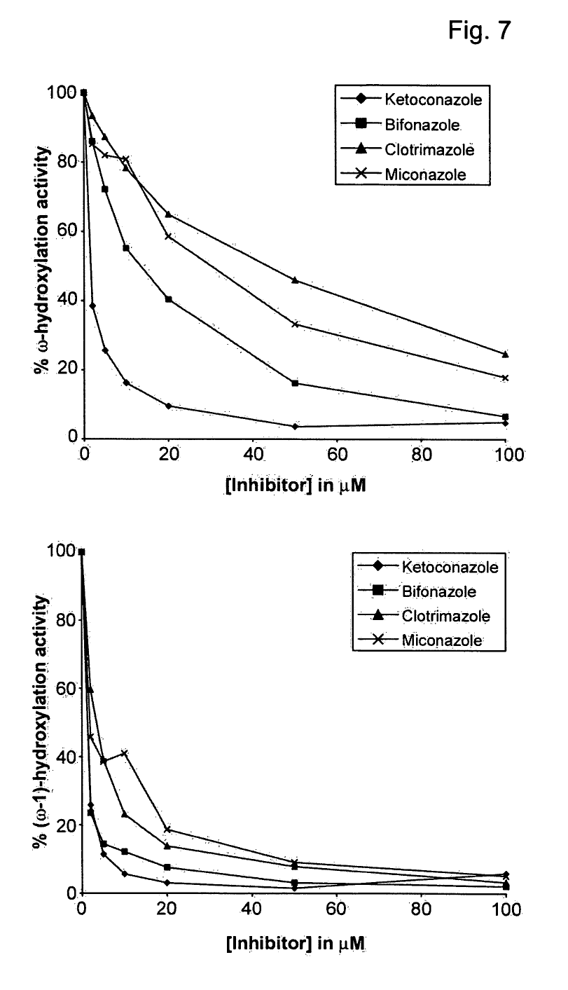

D: The effect of the phytanic acid concentration on the formation of omega-hydroxyphytanic acid was determined using the optimum experimental conditions derived from the previous experiments [100 mM potassium phosphate buffer (pH 7.6), 1 mM NADPH]. The ratio methyl-beta-cyclodextrin to phytanic acid was kept constant. All data shown represent means of duplicate experiments with the exception of FIG. 3C, in which 3-4 separate experiments were done with the SD values shown as error bars.

FIG. 4. Effect of different imidazole antimycotics on the omega- and (omega-1)-hydroxylation of phytanic acid. Rat liver microsomes were incubated with phytanic acid in the presence of different concentrations of imidazole derivatives. The inhibitory effect of the imidazole derivatives on the formation of omega-hydroxyphytanic acid (omega-HPA; A) and (omega-1)-hydroxyphytanic acid (B) is shown. The data shown represent means of duplicate experiments.

FIG. 5. GC-MS (SIM mode) chromatograms of extracts of human liver microsomes incubated with phytanic acid in the absence (x) or presence (squares) of NADPH. For comparison, a chromatogram of an extract of rat liver microsomes incubated with phytanic acid in the presence of NADPH (circles) is shown. The peaks are the (M−15)⁺ ions (m/z=457) of trimethylsilylated omega- and (omega-1)-hydroxyphytanic acid (omega- and (omega-1)-HPA).

FIG. 6. Optimization of phytanic acid omega-hydroxylation in pooled human liver microsomes. (A) The effect of different concentrations of methyl-beta-cyclodextrin on the formation of omega-hydroxyphytanic acid (omega-HPA) in rat liver microsomes was determined. The pH dependency of phytanic acid omega-hydroxylation is shown in (B). The hydroxylase assay was essentially performed as in (A), with the exception of the use of a combined buffer containing 50 mM potassium phosphate and 50 mM pyrophosphate and 0.75 mg/ml methyl-beta-cyclodextrin. The effect of the NADPH concentration on phytanic acid omega-hydroxylation in the presence of an NADPH-regenerating system (10 mM isocitrate, 10 mM MgCl₂ and 0.08 U isocitrate dehydrogenase) is shown in (C). The experimental set-up was as described in the Materials and Methods section of Example 2 with the exception of the use of a 100 mM potassium phosphate buffer (pH 7.7) and 0.75 mg/ml methyl-beta-cyclodextrin. The Kₘ for NADPH was 2 μM as determined from the Lineweaver Burke plot (insert). The effect of the phytanic acid concentration on the formation of omega-hydroxyphytanic acid (D) was determined using the optimum experimental conditions derived from the previous experiments (100 mM potassium phosphate buffer (pH 7.7), 1 mM NADPH). The ratio methyl-beta-cyclodextrin/phytanic acid was kept constant. The data shown represent means of duplicate experiments.

FIG. 7. Effect of different imidazole antimycotics on the omega- and (omega-1)-hydroxylation of phytanic acid. Pooled human liver microsomes were incubated with phytanic acid in the presence of different concentrations of imidazole derivatives. The inhibitory effect of the imidazole derivatives on the formation of omega-hydroxyphytanic acid (A) and (omega-1)-hydroxyphytanic acid (B) is shown. The data shown represent means of duplicate experiments.

TABLE 1
Ligands of nuclear receptor family members

| | Nuclear receptors affected |
|---|---|
| Nuclear receptor ligands | by ligands |
| Oestrogens, retinoids | ERs, RARs, RXRs |
| Androgens | AR |
| Fatty acid derivatives | PPARs |
| Vitamin D3 | AR, ER |
| Androgens, corticoids, | AR, ER, GR, MR |
| oestrogens, pregnanes | |
| Retinoic acid | RARs, RXRs |
| Vitamin D | VDR |
| Oxysterols, sterols | LXR, FXR |

TABLE 2
IC50 values for the inhibition of phytanic acid omega- and
(omega-1)-hydroxylation by imidazole derivatives as
calculated from FIG. 7

| | IC50 value | |
|---|---|---|
| | omega-hydroxyphytanic | IC50 value |
| Inhibitor | acid | (omega-1)-hydroxyphytanic |
| Bifonazole | 13 μM | <2 μM |
| Clotrimazole | 43 μM | 3 μM |
| Ketoconazole | <2 μM | <2 μM |
| Miconazole | 30 μM | <2 μM |

*Data represent the mean of duplicate experiments

### REFERENCES

Baudhuin, P., H. Beaufay, Y. Rahman-Li, O. Z. Sellinger, R. Wattiaux, P. Jacques, and C. De Duve. 1964. Tissue fractionation studies. 17. Intracellular distribution of monoamine oxidase, aspartate aminotransferase, alanine aminotransferase, D-amino acid oxidase and catalase in rat-liver tissue. Biochem. J. 92: 179-184.
Bradford, M. M. 1976. A rapid and sensitive method for the quantitation of microgram quantities of protein utilizing the principle of protein-dye binding. Anal. Biochem. 72: 248-254.
Chalmers, R. A. and A. M. Lawson. 1982. Organic acids in man: The analytical chemistry, biochemistry and diagnosis of the organic acidurias, Chapman and Hall, London.
Halpert, J. R. 1995. Structural basis of selective cytochrome P450 inhibition. Annu. Rev. Pharmacol. Toxicol. 35: 29-53.
Zhang, W., Y. Ramamoorthy, T. Kilicarslan, H. Nolte, R. F. Tyndale, and E. M. Sellers. 2002. Inhibition of cytochromes P450 by antifungal imidazole derivatives. Drug Metab Dispos. 30: 314-318.
Maurice, M., L. Pichard, M. Daujat, I. Fabre, H. Joyeux, J. Domergue, and P. Maurel. 1992. Effects of imidazole derivatives on cytochromes P450 from human hepatocytes in primary culture. FASEB J. 6: 752-758.
D. J. Waxman, P450 gene induction by structurally diverse xenochemicals: central role of nuclear receptors CAR, PXR, and PPAR, Arch. Biochem. Biophys. 369 (1999) 11-23.
P. Honkakoski, M. Negishi, Regulation of cytochrome P450 (CYP) genes by nuclear receptors, Biochem. J. 347 (2000) 321-337.
M. Mukherji, N. J. Kershaw, C. J. Schofield, A. S. Wierzbicki, M. D. Lloyd, Utilization of sterol carrier protein-2 by phytanoyl-CoA 2-hydroxylase in the peroxisomal alpha oxidation of phytanic acid, Chem. Biol. 9 (2002) 597-605.

1. A method for at least in part counteracting a disease state in a subject, said disease state involving accumulation of a fatty acid, the method comprising:
administering, to the subject, a compound able to induce and/or upregulate omega-oxidation of said fatty acid, or whose metabolite is able to induce and/or upregulate omega-oxidation of said fatty acid.

2. (canceled)

3. The method according to claim 1, wherein said disease state comprises a peroxisomal and/or mitochondrial fatty acid oxidation deficiency.

4. The method according to claim 1, wherein said disease state comprises diabetes mellitus, medium chain acyl-CoA dehydrogenase deficiency, Refsum disease and/or X-linked adrenoleukoclystrophy.

5. The method according to claim 1, wherein said compound comprises a cytochrome P450 enzyme or a functional part, derivative and/or analogue thereof, and/or a nucleic acid sequence encoding a cytochrom P450 enzyme or a functional part, derivative and/or analogue thereof.

6. The method according to claim 1, wherein said compound capable of upregulating omega-oxidation comprises a compound capable of increasing a cytochrome P450 enzyme's amount.

7. The method according to claim 1, wherein said compound capable of upregulating omega-oxidation comprises a compound capable of enhancing a cytochrome P450 enzyme's omega-hydroxylation capacity.

8. The method according to claim 6, wherein said cytochrome P450 enzyme comprises a human cytochrome P450 enzyme.

9. The method according to claim 8, wherein said human cytochrome P450 enzyme comprises CYP4A11, CYP4F2, CYP4F3A and/or CYP4F3B.

10. The method according to claim 8, wherein said human cytochrome P450 enzyme comprises CYP4F2 and/or CYP4F3B.

11. The method according to claim 1, wherein said compound is capable of inducing and/or enhancing a cytochrome P450-specific promoter.

12. The method according to claim 1, wherein said compound comprises a ligand of a member of the nuclear hormone family.

13. The method according to claim 12, wherein said compound comprises a ligand of PPARα, LXR, FXR, PXR, and/or CAR.

14. The method according to claim 1, wherein said compound comprises a fibrate, rifampicin, and/or phenyloin.

15. The method according to claim 1, wherein said compound comprises bezafibrate, fenofibrate, gemfibrozil, and/or ciprofibrate.

16. The method according to claim 1, wherein said compound comprises a compound as depicted in Table 1.

17. The method according to claim 1, wherein said fatty acid comprises phytanic acid and/or C26:O.

18. (canceled)

19. A method for determining whether and/or to what extent a compound is capable of inducing and/or upregulating omega-oxidation of a fatty acid, comprising:
providing a non-human animal suffering from, or at risk of suffering from, a disease involving accumulation of said fatty acid with said fatty acid and with said compound,
determining the extent of accumulation of said fatty acid in said animal, and
comparing said extent of accumulation with the extent of accumulation of said fatty acid in the same kind of non-human animal which is not, or to a significantly lesser extent, provided with said compound.

20. The method according to claim 19, wherein said fatty acid comprises C26:O or phytanic acid.

21. (canceled)

22. (canceled)

23. A method for at least in part treating Refsum disease, the method comprising:
administering, to a subject suffering from, or at risk of suffering from, Refsum disease, a compound able to increase the amount and/or the omega-hydroxylation capacity of CYP4A11, CYP4F2, CYP4F3A, and/or CYP4F3B, or whose metabolite is able to increase the amount and/or the omega-hydroxylation capacity of CYP4A11, CYP4F2, CYP4F3A, and/or CYP4F3B.

24. A method for at least in part treating X-linked adrenoleukodystrophy, the method comprising:
administering to a subject suffering from, or at risk of suffering from, X-linked adrenoleukodystrophy, a compound able to increase the amount and/or the omega-hydroxylation capacity of CYP4F2 and/or CYP4F3B, or whose metabolite is able to increase the amount and/or the omega-hydroxylation capacity of CYP4F2 and/or CYP4F3B.

25. A kit comprising:
a first compound able to increase a first enzyme's amount and/or the omega-hydroxylation capacity, the first enzyme selected from the group consisting of CYP411, CYP4F2, CYP4F3A, CYP4F3B, and combinations of any thereof, or whose metabolite is able to increase the first enzyme's amount and/or the omega-hydroxylation capacity, and
a second compound able to increase a second enzyme's amount and/or omega-hydroxylation capacity, the second enzyme selected from the group consisting of CYP4A11, CYP4F2, CYP4F3A, CYP4F3B, and combinations of any thereof, or whose metabolite is able to increase the second enzyme's amount and/or the omega-hydroxylation capacity.

26. A kit comprising:
a first compound able to increase CYP4F3B's amount and/or the omega-hydroxylation capacity, or whose metabolite is able to increase CYP4F3B's amount and/or omega-hydroxylation capacity, and
a second compound able to increase CYP4F3B's amount and/or omega-hydroxylation capacity, or whose metabolite is able to increase CYP4F3B's amount and/or omega-hydroxylation capacity.

27. The method according to claim 7, wherein said cytochrome P450 enzyme comprises a human cytochrome P450 enzyme.
